(19) United States
(12) Patent Application Publication
(10) Pub. No.: US 2023/0225644 A1
(43) Pub. Date: Jul. 20, 2023

(54) REUSABLE AND ELECTROCHEMICALLY ACTIVE DEVICE FOR MEASUREMENT OF CONCENTRATION OF BIOANALYTES

(71) Applicant: PATHSHODH HEALTHCARE PVT. LTD., Bengaluru (IN)

(72) Inventors: Vinay Kumar, Bengaluru (IN); Navakanta Bhat, Bengaluru (IN)

(73) Assignee: PATHSHODH HEALTHCARE PVT. LTD., Bengaluru (IN)

(21) Appl. No.: 18/156,950

(22) Filed: Jan. 19, 2023

(30) Foreign Application Priority Data
Jan. 19, 2022 (IN) 202241003075

(51) Int. Cl. A61B 5/1468; A61B 5/145

(52) U.S. Cl. CPC A61B 5/1468; A61B 5/14532; A61B 5/14507

(57) ABSTRACT

A reusable and electrochemically active device 100 is provided comprising, detachable electrode arrangement including working electrodes 101a, 101b, 101c, 101d and a counter electrode 102 that are functionalised with selected electrochemically active receptor(s) that can interact with at least a target bioanalyte. Fluid transportation channels 108a, 108b, 108c, 108d are formed, to receive biological samples with the at least target bioanalyte and for further transportation to the selected electrode arrangement, preferably in a sequential manner, for measuring the concentrations of the target bioanalytes. Used working electrodes 101a, 101b, 101c, 101d and a partial portion of the counter electrode 102 are detachable from the device 100. Insulating members 113a, 113b, 113c, 113d, 113e, 113f, 113g are disposed on the electrode arrangement such that the integrity of the electrical connectivity of the remaining electrode arrangement is retained even after the detachment of the used working electrodes 101a, 101b, 101c, 101d and a portion of the counter electrode 102. The present invention also provides a point-of-care biosensor 300 and a method to electrochemically measure the concentrations of multiple target bioanalytes and a single bioanalyte repeatedly.

### FIELD OF INVENTION

The present invention relates to a reusable electrochemically and active device for measuring concentrations of bioanalytes in biological samples. The present invention particularly relates to a reusable and electrochemically active device with a detachable electrode arrangement and a method for measuring concentrations of bioanalytes in biological samples.

### BACKGROUND OF THE INVENTION

Monitoring of concentrations of bioanalytes, such as glucose, albumin, haemoglobin, creatinine etc., in a biological sample such as blood or urine is an important part in the management of medical indications that are caused the changes in the concentrations, which are beyond the accepted levels.

Electrochemical detection and measurement of concentrations of such bioanalytes is generally performed by loading a selected biological sample on a test strip (a miniature electrochemical cell) that is configured with a selective chemistry and tested electrochemically, to determine the concentrations of the bioanalytes.

However, such test strips are required to be disposed of after single use, to prevent cross-contamination.

In addition, such a disposable test strip also does not enable the testing of multiple bioanalytes or a single bioanalyte multiple times, on a single test strip.

Therefore, there is a need to develop a reusable and electrochemically active device, which can measure not only the concentrations of multiple bioanalytes in biological samples but also enables repeated measurement of concentrations of a single bioanalyte, by avoiding cross-contamination.

### OBJECTS OF THE PRESENT INVENTION

The present invention is made to solve the above-mentioned problems and has for its object to provide a reusable and electrochemically active device with a detachable electrode arrangement, to electrochemically measure not only the concentrations of multiple target bioanalytes but also to measure, repeatedly, concentration of selected target bioanalyte, in biological samples.

An object of the present invention is to provide a reusable and electrochemically active device with detachable fluid transportation channels for introducing and transporting biological samples to the electrode arrangement.

A further object of the present invention is to provide a reusable and electrochemically active device with fluid transportation channels with a marker to facilitate a sequential loading of biological samples with target bioanalytes.

Yet another object of the present invention is to provide a point-of-care biosensor to measure and display the concentrations of bioanalytes in biological samples, using the device of the present invention.

It is also an object of the present invention to provide a method to electrochemically measure not only the concentrations of multiple target bioanalytes using the device of the present invention, but also to measure, repeatedly, concentration of selected target bioanalyte, in biological samples, by using a single reusable and electrochemically active device of the present invention.

### BRIEF DESCRIPTION OF THE DRAWINGS

FIG. 1 is a schematic illustration of the reusable and electrochemically active device with detachable electrode arrangement of two working electrodes with fluid transportation channels.

FIG. 2 is a schematic exploded view of the reusable and electrochemically active device as shown in FIG. 1.

FIG. 3 is a schematic illustration of the reusable and electrochemically active device with detachable electrode arrangement of four working electrodes with fluid transportation channels.

FIG. 4 is a schematic illustration of the reusable and electrochemically active device with fluid transportation channels having horizontal and angular orientations.

FIG. 5 is a schematic illustration of the reusable and electrochemically active device with fluid transportation channels arranged in a combination of horizontal and vertical orientations.

FIG. 6 is a schematic illustration of the reusable and electrochemically active device with set of fluid transportation channels that are connected to the each of the working electrodes.

FIG. 7 is a schematic illustration of the device holder of the present invention that is connected to the reusable and electrochemically active device.

FIG. 8 is a schematic illustration of the point-of-care device of the present invention that is connected to the reusable and electrochemically active device.

FIG. 9 is a schematic illustration of the internal architecture of the point-of-care device.

FIG. 10 is a flow chart depicting broad steps of the method of the present invention.

FIG. 11 is an exemplary linearity plot of redox current versus blood glucose concentrations in two fluid transportation channels the reusable and electrochemically active device.

FIGS. 12(a)-(d) are exemplary linearity plots of redox current versus blood glucose concentrations in four fluid transportation channels the reusable and electrochemically active device.

FIG. 13(a)-(b) are exemplary linearity plots of Redox current Vs blood glucose in fluid transportation channel-1 and Redox current Vs hemoglobin in fluid transportation channel-2.

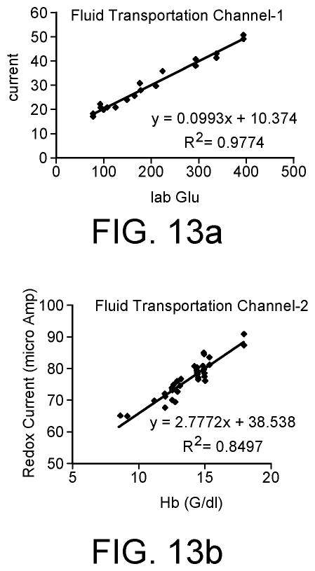

### SUMMARY OF THE PRESENT INVENTION

Accordingly, the present invention provides a reusable and electrochemically active device comprising, detachable electrode arrangement including working electrodes and a counter electrode that are functionalised with selected electrochemically active receptor(s) that can interact with at least a target bioanalyte. Fluid transportation channels are formed, to receive biological samples with the at least target bioanalyte and for further transportation to the selected electrode arrangement, preferably in a sequential manner, for measuring the concentrations of the target bioanalytes. The working electrodes, the fluid transportation channels and a partial portion of the counter electrode are detachable, after their use, from the device while maintaining the integrity of the electro-chemical nature of other working electrodes, counter electrode and fluid transportation channels, by providing insulating members that are disposed on the electrode arrangement. The present invention also provides a point-of-care biosensor and a method to electrochemically measure and display the concentrations of multiple target bioanalytes and of a single bioanalyte repeatedly.

### DETAILED DESCRIPTION OF THE INVENTION

In order to understand the salient principles underlying the invention, reference will now be made to the embodiments illustrated in the accompanied drawings and a specific language is used to describe those illustrated embodiments. It is therefore to be understood that no limitation of the scope of the invention is intended. Alterations and modifications to the illustrated device and method and further applications of the principles of the invention as illustrated therein, as would normally occur to one skilled in the art to which the invention relates are contemplated, are desired to be protected. In particular, although the invention is described in terms of measuring the concentrations of some of the selected bioanalytes, it is contemplated that the device and method of the present invention can be used to measure the concentrations of other bioanalytes present in various biological samples. It is also understood that such alternative embodiments may require certain adaptations to the embodiments described herein that would be obvious to those skilled in the relevant art.

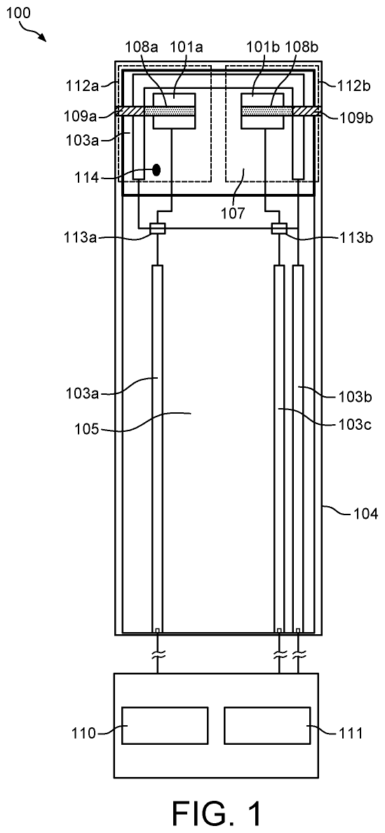

Although the reusable electrochemically device, point-of-care biosensor and method of the present invention may be used with test strips having a wide variety of designs and made with a wide variety of construction techniques, a typical electrochemical test strip (reusable electrochemically device) of the present invention is illustrated in FIG. 1.

The reusable and electrochemically active device 100 as shown in FIG. 1 comprises a bottom substrate 104, which acts as a base on which other constituents of the device 100 are fabricated. The substrate 104, in this embodiment is exemplarily shown as an elongated rectangular structure. However, it is understood here that the substrate 104 can take other shapes such as square, circular depending on the shape and configuration of other related coupled devices such as a biosensor that holds the device 100. The substrate 104 can be made of any suitable rigid or flexible material that is suitable for the incorporation of patterned electrodes. For instance, materials such as polyvinylchloride (PVC), polyethylene terephthalate (PET), polymethylmethacrylate (PMMA), epoxy fiber composites, polyamides composites, and paper can be used as preferred materials for the substrate 104. Whereas, the preferred rigid materials for the substrate 104 can be ceramic, glass or any other like materials. In any case, the selection of suitable material for the substrate 104 is made to ensure that the substrate 104 can not only provide a desirable strength and flexibility but also can act as an electrical insulator. Advantageously the substrate 104, considering the applications of the invention, is hydrophilic in nature to prevent percolation of the biological sample, when it comes in physical contact with the substrate 104. The surface of the substrate 104 is generally provided with a smooth texture. However, the substrate 104 can also be provided with a rough surface and/or with cavities or wells. The edges of the substrate 104 are also provided with suitable profiles, such as tapered or curved, to facilitate an easy ingress into and egress out of a biosensor that is used for the measurement of bioanalytes. The top surface of substrate 104 is coated with a 50 nm conductive (gold) layer (by sputtering or vapor deposition, for example). An electrode arrangement is then patterned in the conductive layer, preferably by a laser ablation process.

In the device 100 as particularly shown in FIG. 1, the electrode arrangement is constructed with a pattern of two working electrodes 101a, 101b, along with a counter electrode 102, which are connected to electrically conductive tracks 103a, 103b, 103c and disposed on the substrate 104. In the illustrative electrode arrangement as shown in FIG. 1, the electrodes 101a, 101b act as working electrodes and whereas the electrode 102 acts as a counter electrode and as well as a reference electrode. The material for the working electrodes 101a, 101b is electrochemically active materials selected from metals, organic materials or alloys, such as gold, platinum, mercury, carbon, glassy carbon and graphite. The preferred material for the counter electrode 102 is silver (Ag), a silver chloride (AgCl), silver/silver chloride (Ag/AgCl) or saturated calomel electrode (SCE), where the potential of the electrode does not change with time.

The conductive tracks 103a, 103b, 103c are formed by any patterning method, such as, screen printing, lithography, thermal evaporation, sputtering, laser patterning, preferably screen-printing. However, the number of the conductive tracks is variable and depends on the number of the working electrodes and the number of capillary channels that are preferred for the device 100 (single test strip).

The material for the conductive tracks 103a, 103b, 103c is selected from electrically conductive materials such as copper, aluminum, gold, silver, platinum, carbon, or any other suitable electrically conducting material or alloys of these materials. The material for the conducting tracks 103a, 103b, 103c can also be selected from electrochemically active materials such as gold, platinum, mercury, carbon, glassy carbon and graphite. The conducting tracks 103a, 103b, 103c are used to establish an electrical connection with other devices such a device holder, a biosensor and a system (as hereinafter described), for measuring and displaying the concentrations of desired bioanalytes, in biological samples.

The working electrodes 101a, 101b and the counter electrode 102 are overlaid on the substrate 104 and connected to the conducting tracks 103a, 103b, 103c, as shown in FIG. 1. The working electrodes 101a, 101b and the counter electrode 102 are electrically connected to the conducting tracks 103a, 103b, 103c. The representative material for the working electrodes 101a, 101b is selected from metals, which are electrochemically active, such as gold, platinum, mercury, carbon, glassy carbon and graphite.

The working electrodes 101a, 101b are functionalised with an electrochemically active receptor, which is a chemical substance or a reagent that can bind with a target bioanalyte present in a biological sample.

In other words, the working electrodes 101a, 101b are adapted to be in chemical contact with the electrochemically active receptor.

Advantageously, the initiation of chemical contact of the electrochemically active receptor with the working electrodes 101a, 101b is performed by preparing a solution of the electrochemically active receptor and the prepared solution is dispensed on the working electrodes 101a, 101b electrodes or on a membrane (not shown in the drawings) that is arranged on the working electrodes and dried to form a solid chemical layer on the working electrodes 101a, 101b or the membrane. Alternately, the receptor solution is pre-mixed with the biological sample and dispensed on the working electrodes 101a, 101b or on the membrane.

The initiation of chemical contact of the receptor with the electrodes the working electrodes 101a, 101b can also be performed by preparing a receptor solution separately and dispensing the prepared solution on the working electrodes 101a, 101b or on the membrane.

A spacer 105, is selected, which is formed from an electrically insulating material, as a layer over the electrode arrangement, such that a minimal spacing of the working and counter electrodes is facilitated. The thickness of this spacer layer generally ranges from about 1 to 500 μm, usually from about 102 to 153 μm. The spacer 105 may be fabricated from any convenient material, where representative suitable materials include PET, PETG, polyimide, polycarbonate and the like, where the surfaces of the spacer 105 may be treated so as to be adhesive with respect to the electrode arrangement such that the spacer 105 is overlaid on the electrode arrangement of the substrate 104.

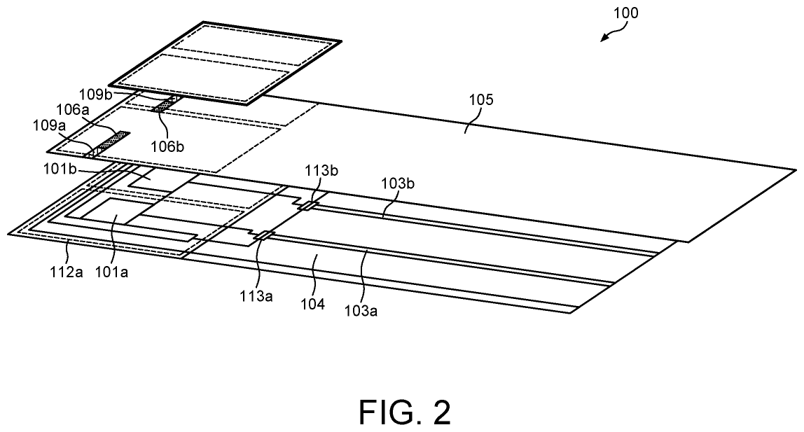

Openings 106a, 106b are formed on the spacer 105, preferably by laser etching. Accordingly, the openings 106a, 106b extend from the peripheries of the substrate 104 and extend over the working electrodes 101a, 101b, such that the at least portions of the working electrodes 101a, 101b and the counter electrode 102 are exposed, as shown in FIGS. 1 and 2. The openings 106a, 106b are with narrow dimensions that are reciprocal to the dimensions of the electrode arrangement and preferably in the range of 0.1 mm to 10 mm.

A laminating member 107 is arranged on the spacer 105, such that the openings 106a, 106b are covered and expose the underlying electrode configuration, as shown in FIG. 1. The laminating member 107 made of a representative hydrophilic material, selected from one of cellulose acetate, polyamide, nylon, polyvinylidene fluoride (PVDF), polystyrene, polypropylene, polyether, polymers incorporated with inorganic or organic nanomaterials.

Fluid transportation channels 108a, 108b are formed in the intervening spaces between the openings 106a, 106b and the laminating member 107, as particularly shown in FIG. 2. The fluid transport channels 108a, 108b are therefore, arranged to receive the biological sample and transport to the working electrodes 101a, 101b and the counter electrode 102 through capillary action.

Seal elements 109a, 109b are preferably are provided at the terminal ends of the fluid transportation channels 108a, 108b. The seal elements are preferably made of flexible polymer material and are configured to adhere to the laminating member 107 on one side and the bottom portion of the substrate 104, such that the seal elements close the openings of the fluid transportation channels 108a, 108b. The seal elements 109a, 109b can also be suitably adapted for repeated opening and closing of the fluid transportation channels 108a, 108b, to regulate the ingress of biological sample into the fluid transportation channels 108a, 108b and prevent the contamination of the biological sample from surrounding conditions.

A voltage source 110 is configured to be coupled to the working electrodes 101a, 101b through the conducting tracks 103a, 103b, 103c and adapted to apply a redox voltage.

A current sensor 111 is configured to be coupled to measure a redox current from the functionalized working electrodes 101a, 101b, upon interacting with the target bioanalyte. The measured redox current is usable to obtain a concentration of the target bioanalyte, by correlating the measured redox current with a reference concentration of the target bioanalyte.

The voltage source 110 and the current sensor 111 are also configured to automatically detect the unused working electrodes 101a, 101b of the electrochemically active device 100.

It is understood here that the voltages source 110 and the current sensor 111 are preferably connected externally and may not be integral to the reusable and electrochemically active device 100.

The electrode arrangement, in particular, the working electrodes 101a, 101b and the counter electrode 102 are detachable, after use, individually, from the substrate 104, for disposal, without affecting the electrode arrangement that is remaining on the substrate 104. For instance, if the working electrode 101a is used for testing a biological sample for measuring the concentration of a target bioanalyte, this working electrode 101a can be detached from the substrate 104, along with a partial portion of the counter electrode 102 and the fluid transportation channels 108a, 108b, which are involved in the measurement of the concentration of the target bioanalyte.

In order to enable an easy detachment of the required section(s) of the electrode arrangement from the substrate 104, the designated section of the substrate 104 is defined preferably by a series of blind perforations or indentations 112. If preferred, the corresponding section(s) 112a, 112b of the laminating member 107, spacer 105 and the electrode arrangement are also defined by blind perforations or indentations, such that the designated sections can be easily detached by a user either manually or by using a clipping tool, subsequent to the use of one of the working electrodes 101a, 101b and a partial portion of the counter electrode 102. Therefore, the designated section(s) of the substrate 104 for the detachment include the working electrodes 101a and 101b, the counter electrode 104 and the fluid transportation channels 108a and 108b, which are involved in the measurement of the concentrations of the target bioanalytes.

Electrical insulating members 113a, 113b are arranged for the conducting tracks 103a, 103b, as shown in FIG. 1, to facilitate the electrical connectivity of counter electrode 102 with conducting track of the counter electrode 103c, even when a section or a portion of the electrode arrangement is detached from the substrate 104, thus maintaining the electro-chemical integrity of the device 100, in particular with the counter electrode 102. In other words, the electro-chemical integrity of the remaining working electrode 103b, the partial portion of the counter electrode 102 and the fluid transportation channel 108b and the conducting tracks 103a, 103b, 103c, is maintained, for further reuse.

The arrangement of electrical insulating members 113a, 113b, on the conducting tracks 103a, 103b, as shown in FIG. 1, facilitate the repeated use of the same device 100, for measuring the concentrations of the multiple target bioanalytes from different biological samples and also for repeated measurement of a single target bioanalyte, in different biological samples and also ensures prevention of contamination of the unused electrode arrangement.

The selected target bioanalyte(s) include bioanalytes such as glucose, proteins, peptides, enzymes, antigens and antibodies, which interacts with the at least target bioanalyte. It is understood here that the interaction between the electrode arrangement that is functionalised with an electrochemically active receptor and the target bioanalytes is generally a physical binding interaction or a chemical reaction.

Accordingly, the reusable and electrochemically active device 100 as shown in FIGS. 1 and 2 comprises the electrode arrangement including working electrodes 101a, 101b, the counter electrode 102 that are functionalized with an electrochemically active receptor corresponding to the at least target bioanalyte present in the at least biological sample and the conducting tracks 103a, 103b, 103c that arranged on the substrate 104. The spacer 105 is disposed on the substrate 104 such that it overlays on the electrode arrangement. The openings 106a, 106b are formed on the spacer 105 to expose at least the sections or portions of the functionalized working electrodes 101a, 101b and the counter electrode 102. The laminating member 107 is laid on the functionalized working electrodes 101a, 101b and the counter electrode 102 such that the intervening spaces between the openings 106a, 106b and the inner portion of the laminating member 107, thus forming the fluid transportation channels 108a, 108b that are adapted to receive the at least biological sample with the at least target bioanalyte and transport to the functionalized working electrodes 101a, 101b and the counter electrode 102 by capillary action. The voltage source 110 is configured to be coupled to the electrode arrangement and adapted to apply a redox voltage to the functionalised working electrodes 101a, 101b and the counter electrode 102. The current sensor 111 is provided and configured to be coupled to measure a redox current from the functionalized working electrodes 101a, 101b, upon interacting with the at least target bioanalyte. The measured redox current is then usable to obtain the concentration of the at least target bioanalyte in at least the biological samples, by correlating the measured redox current with a reference concentration of the at least target bioanalyte.

In an embodiment of the present invention, the used working electrodes 101a, 101b, the partial portion of the counter electrode 102 and the fluid transportation channels 108a, 108b are detachable, from the substrate 104, by a user such that other portions of the device 100 is not contaminated by the presence of any residual samples.

In yet another embodiment of the present invention the insulating members 113a, 113b are disposed on the conducting tracks 103a, 103b such that the electrical connectivity of the electrode arrangement, in particular the electrical connectivity of the partially detached counter electrode 102, is retained even after the detachment of one of the used working electrodes 101a, 101b, along with the partial portion of the counter electrode 102 and one of the used fluid transportation channels 108a, 108b, from the reusable and electrochemically active device 100.

In yet another aspect of the present invention, the reusable and electrochemically active device 100, comprises a marker or a sequence indicator 114, for instance a symbol or numerals, is disposed at a pre-determined working electrode, which in this embodiment is the working electrode 101a, to indicate the commencement of sequence of introducing of biological samples into the fluid transportation channels 108a, 108b for onward transportation to the respective electrodes 101a, 101b.

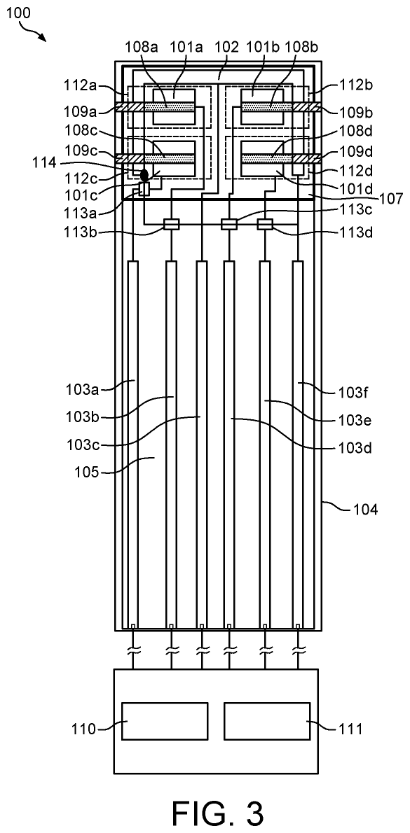

In another aspect of the present invention, the reusable and electrochemically active device 100 comprises an electrode arrangement including a plurality of functionalised working electrodes 101a, 101b, 101c, 101d, as shown in FIG. 3. The constructional aspects of the electrode arrangement are generally as described above, in respect of the two-working electrode configuration, with suitable adaptions in the arrangement of the insulating members 113a, 113b, 113c, 113d, such that the integrity of the electrical-chemical functionality of the electrode arrangement and the fluid transportation channels is maintained, even after the detachment of any of the used electrodes from the reusable and electrochemically active device 100.

In the electrode arrangement as illustrated in FIG. 3, fluid transportation channels 108a, 108b, 108c, 108d are formed for the introduction of biological samples with target bioanalytes. The introduced biological samples with at least the target bioanalyte, are then transported to the corresponding working electrodes 101a, 101b, 101c, 101d, which are in fluid communication with the fluid transportation channels 108a, 108b, 108c, 108d, for the measurement of concentrations of target bioanalytes. In this configuration, it is therefore, possible to measure target bioanalytes from different biological samples from different working electrodes 101a, 101b, 101c, 101d. This exemplary configuration also enables variable functionalisation of the working electrodes, by selecting electrochemically active receptors, which are selective to the desired target bioanalytes. In other words, each of the working electrodes 101a, 101b, 101c, 101d can be used to measure concentrations of different target bioanalytes present in the biological samples. This configuration also enables measurement of concentrations of the same target bioanalyte repeatedly from different biological samples.

Therefore, in this embodiment, the reusable and electrochemically active device 100 comprises the electrode arrangement including the functionalized working electrodes 101a, 101b, 101c, 101d, the counter electrode 102 that are in fluid communication with plurality of fluid transportation channels 108a, 108b, 108c, 108d. The insulating members 113a, 113b, 113c, 113d are disposed to connect the counter electrode 102, the working electrodes 101a, 101b, 101c, 101d and the conducting tracks 103a, 103b, 103c, 103d, such that the electrical connectivity of the electrode arrangement is retained even after detachment at least one of the used working electrodes 101a, 101b, 101c, 101d, partial portions of the counter electrode 102 and at least one of the used fluid transportation channels 108a, 108b, 108c, 108d, from the reusable and electrochemically active device 100. In other words, the electro-chemical integrity of the remaining working electrodes 101b, 101c, 101d, the counter electrode 102, the partial portion of the counter electrode 102 and the fluid transportation channel 108b, 108c, 108d and the conducting tracks 103a, 103b, 103c, 103d, 103e, 103f is maintained, for further reuse.

In yet another aspect of the present invention, the reusable and electrochemically active device 100, as particularly shown in FIG. 3, comprises a marker or a sequence indicator 114, for instance a symbol or numerals, is disposed at a pre-determined working electrode, which in this embodiment is the working electrode 101a, to indicate the commencement of sequence of introducing of biological samples into the fluid transportation channels 108a, 108b, 108c, 108d for onward transportation to the respective electrodes 101a, 101b, 101c, 101d.

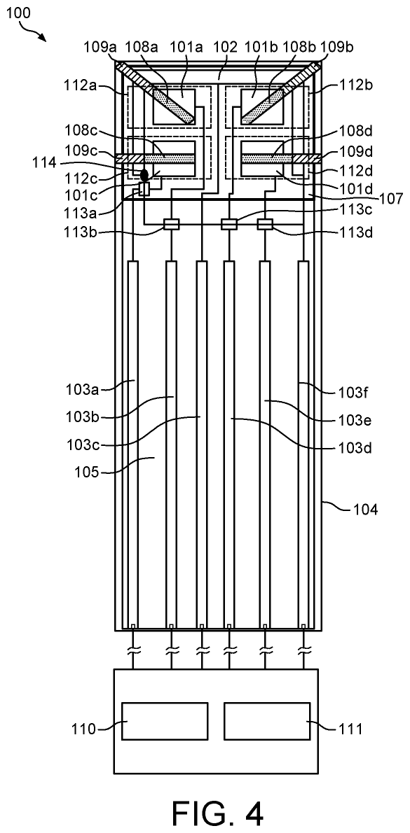

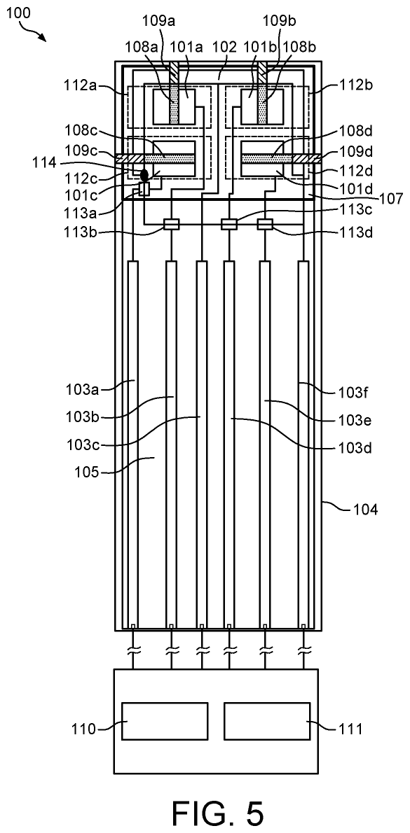

Now, the preferred embodiments, pertaining to the various positional orientations of the fluid transportation channels 108a, 108b, 108c, 108d that are in fluid communication with the working electrodes 101a, 101b, 101c, 101d are described, by referring to FIGS. 3, 4 and 5.

The fluid transportation channels 108a, 108b, 108c, 108d are arranged horizontally along the planar surface of the working electrodes 101a, 101b, 101c, 101d, with their terminal ends extending to the side peripheries of the substrate 104, to facilitate introduction of biological samples, as shown in FIGS. 1, 2 and 3.

The fluid transportation channels 108a, 108b, 108c, 108d can also be arranged at various other orientation positions, such as horizontal, angular and vertical orientations. The different orientations of the fluid transportation channels 108a, 108b, 108c, 108d, facilitate ease of assembly of the device 100 in a holder or biosensor for the measurement of concentration ns of bioanalytes in biological samples. This arrangement also facilitates multi-directional and multi-locational access points for introducing the biological samples.

In the embodiments as shown in electrical insulating members 113a, 113b, 113c, 113d are provided to the conducting tracks 103a, 103b, 103c, 103d as shown in FIGS. 3-5, to maintain the integrity of the electro-chemical functionality of the device 100, even when a section or sections of the electrode arrangement, including working electrodes and the counter electrode, is detached from the substrate 104.

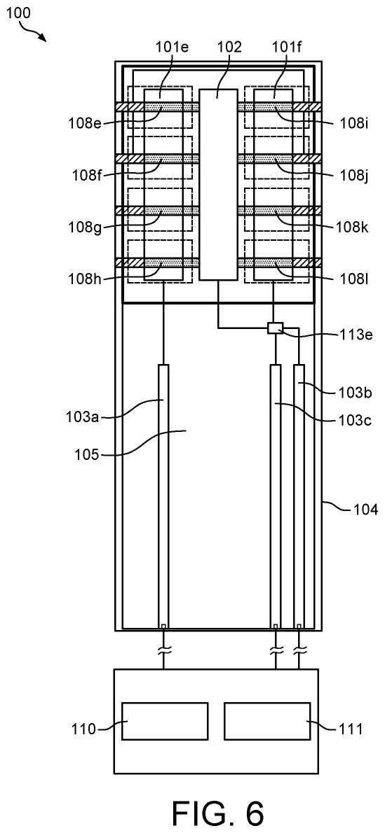

In yet another aspect of the present invention, the reusable and electrochemically active device 100, as shown in FIG. 6, comprises a set of fluid transportation channels 108e, 108f, 108g, 108h that are in fluid communication with the functionalized working electrode 101e, which is larger is size and another set of fluid transportation channels 108i, 108j, 108k, 108l are in fluid communication with the functionalized working electrode 101f, which is also larger in size. The counter electrode 102 is disposed in between the functionalized working electrodes 101e, 101f. In this arrangement each of the sections of the fluid transportation channels 108e, 108f, 108g, 108h, 108i, 108j, 108k, 108l, along with respective portions of the working electrodes 101e, 101f, are detachable from the substrate 104 subsequent to their use.

Electrical insulating member 113e is provided to the conducting track 103b as shown in FIG. 6, to maintain the integrity of the electro-chemical functionality of the device 100, even when a section or sections of working electrodes 101e, 101f of the electrode arrangement, is or are detached from the reusable and electrochemically active device 100, after their use.

In yet another aspect of the present invention, the reusable and electrochemically active device 100, as shown in FIGS. 1-5, sealing members 109a, 109b, 109c, 109d are connected to the fluid transportation channels 108a, 108b, 108c, 108d. The sealing members 109a, 109b, 109c, 109d are preferably flexible sealing strips that are used to act as closure means to close and open the openings of the fluid transportation channels 108a, 108b, 108c, 108d, whenever needed and in particular to keep the channels closed soon after the completion of transmission of the biological samples to the working electrodes. The sealing members 109a, 109b, 109c, 109d are advantageously made of flexible polymer materials, which are inert in nature such that they can be adhered to the substrate 104 and the laminating member 107, while operating them for closing and opening of the fluid transportation channels 108a, 108b, 108c, 108d.

In a particular embodiment of the reusable and electrochemically active device 100, as shown in FIG. 6, sealing members 109e, 109f, 109g, 109h, 109i, 109j, 109k, 109l are connected to the fluid transportation channels 108e, 108f, 108g, 108h, 108i, 108j, 108k, 108l.

The voltage source 110 and the current sensor 111 the reusable and electrochemically active device 100, as illustrated in FIGS. 1-6 are configured automatically detect the used and unused working electrodes 101a, 101b, 101c, 101d of the electrochemically active device 100.

The voltage source (110) and the current sensor (111) are disposed and adapted to automatically detect the used and unused working electrodes (101a, 101b, 101c, 101d) and corresponding sections of counter electrode (102) of the electrochemically active device (100).

The exemplary target bioanalytes, the concentrations of which are measured in the biological samples, using the reusable and electrochemically active device 100, include glucose, proteins, peptides, enzymes, antigens and antibodies. The selection of the electrochemically active receptors, which are used to functionalise the working and counter electrodes, is based their interactive nature, which includes binding and chemical reactive nature with desired target bioanalytes.

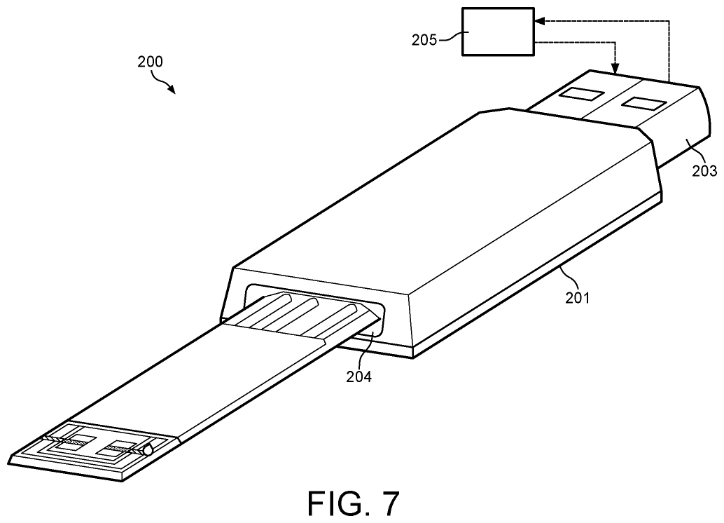

The device holder 200 comprises, a device detection module 202 with suitable internal circuitry that is arranged in a housing 201 for detecting the reusable and electrochemically active device 100. A device insertion port 204, is connected to housing 201, to permit the connectivity of the reusable and electrochemically active device 100 to the device holder 200. A USB connector 203 is arranged at one end of the housing 201, for enabling a connectivity with an external processing resource 205, which for instance can be a hand-held computing device or communicating device with a processor, for measurement of concentration of at least a target bioanalyte, as shown in FIG. 7. The device holder 200 may also be provided with data storage, signal conditioning module 202 with the voltage source 110 and current sensor 111 (as shown in FIGS. 1-6) and data acquisition modules, to identify the type of bioanalyte(s) that is stored on the reusable and electrochemically active device 100. Therefore, the device holder 200 is used to collect and retain the biological samples for subsequent testing. The device holder 200 further enables a user to insert the holder 200 into a computing device for the measurement of concentration of target bioanalytes.

In this aspect, the device holder 200 with the holder housing 201 includes the device detection and signal conditioning module and the USB connector 203 that is adapted to be connected to the external electronic processing resource 205. The reusable and electrochemically active device 100 is adapted to be connected to the device insertion port 204 and to the external electronic processing resource 205. The reusable and electrochemically active device 100 is adapted to receive the at least biological sample with the at least target bioanalyte, through the at least unused fluid transportation channels 108a, 108b, 108c, 108d, 108e, 108f, 108g, 108h, 108i, 108j, 108k, 108l, and transported to the at least unused working electrode 101a, 101b, 101c, 101d, 101e, 101f and the unused portion of the counter electrode 102, after establishing a connectivity with the external electronic processing resource 205, to measure the concentration of the target bioanalyte(s). The biological samples that are preferred for measuring the target bioanalytes are blood or urine.

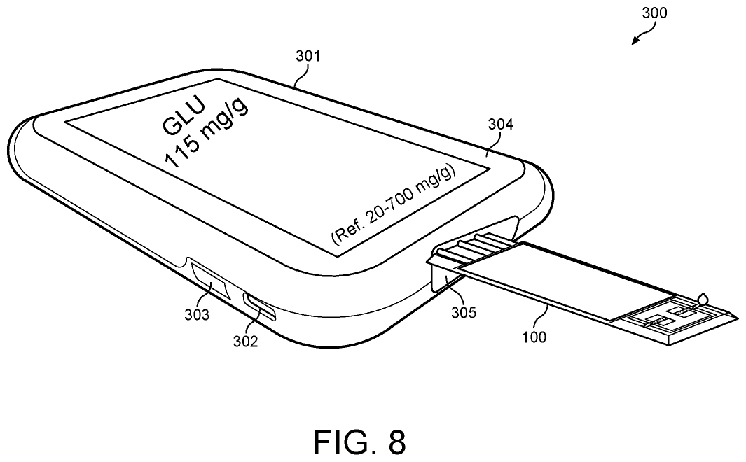

The preferred embodiments of the point-of-care biosensor 300 of the present invention, for measuring the concentration of at least a target bioanalyte in a biological sample, using the reusable and electrochemically active device 100 are now described by referring to FIG. 8. The point-of-care biosensor 300 comprises a housing 301. A micro USB 302 and micro SD card 303, are arranged in the housing 301. The micro USB 302 is used to charge the biosensor 300 and micro SD card is used as a storage device. The housing 301 is also provided with display member 304, which can be an LCD, LED, OLED, OMLED, TFT or any other such display devices, including touch-sensitive devices. A device insertion port 305 is provided in the housing 301. Metallic contacts of the device insertion port 305 engage the reusable and electrochemically active device 100 electrically. In other words, the insertion port 305 is provided to receive the reusable and electrochemically active device 100, through the electrode arrangement of the reusable and electrochemically active device 100. The point-of-care biosensor 300 is provided to facilitate a user to use the reusable and electrochemically active device 100, in a simple way, along with the point-of-care biosensor 300. The reusable and electrochemically active device 100 loaded with biological sample(s) is initially inserted into the point-of-care biosensor 300 and loaded with a selected biological sample, in reduced volume, in the range of 1-300 μL, which entails a minimum invasive means in collecting the biological sample.

The user is also at liberty to use the biosensor 300 at a room temperature and without concerning about other environmental factors such as humidity, temperature variation and storage conditions. The user by using the biosensor 300 is able to measure the concentration levels of the selected target bioanalytes, in a substantially shorter period of time, since the bioanalyte binds the receptor, instantaneously. The user is provided with an instantaneous and accurate display of the concentration of the selected target bioanalytes on the display member 304, since the inherent binding nature of target bioanalyte is used in the biosensor 300 to measure the concentration levels. By using the biosensor 300 of the present invention, the user is enabled to use the biosensor without a need for active preparation of the biological sample before it is tested.

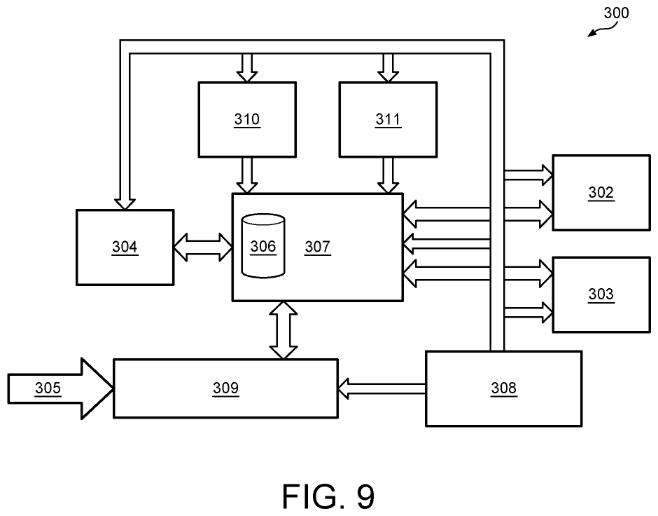

Now, referring to FIG. 9, an internal electronic hardware architecture of the point-of-care biosensor 300 is described. A database member 306 is provided in the housing 301, to store standard values of redox current and bioanalyte concentration of desired bioanalytes that are present in the biological samples. The database 306 also incorporates the data pertaining to historical and current data of concentrations of the bioanalytes. The executables that are required to perform the various functions of the biosensor 300 are stored on a medium of the biosensor 300.

The database member 306 is arranged to store the standard values of concentrations of the target bioanalytes concentrations along with reciprocal redox currents.

A power supply to the biosensor 300 is regulated by a power supply unit 308, which is connected to the biosensor 300. The power supply unit 308 includes both online and offline rechargeable battery with charging circuitry. A signal conditioning and device detection unit 309 is connected to the microcontroller 307 to detect the presence of the device 100 in the biosensor 300 and to apply the redox potential to the electrode arrangement having the selected biological samples with target bioanalytes, through the voltage source and measure the redox current through the current sensor. Therefore, the signal conditioning circuitry of the signal conditioning and device detection unit 309 applies redox current across the conductive lines of the working and counter electrodes of the biosensor 300 and simultaneously measures the redox current for further analysis of concentration of the desired bioanalytes.

Humidity and temperature sensors 310 and 311 are arranged in the housing 301. Once the measurement of the concentration levels of the bioanalyte is completed by the microcontroller 307, the concentration levels are displayed on the display member 304, along with historical data of the concentration levels of the bioanalyte.

The signal conditioning and device detection unit 309 along with the microcontroller 307, perform required operations to identify the available working electrodes for receiving biological samples.

The signal conditioning and device detection unit 309 along with the microcontroller 307, also performs operations to identify the detached portions of electrode arrangement, which are detached from the device after its use.

Therefore, the point-of-care biosensor 300 for measuring concentrations of target bioanalytes in biological samples is provided with the micro USB 302, the micro SD card 303, the display member 304, the device insertion port 305, the database member 306, the voltage source 310 and the current sensor 311, the signal conditioning and device detection unit 309, humidity and temperature sensors 310, 311 are adapted to be connected to the digital controller 307 and disposed in the sensor housing 301 and connected to a power supply unit 308. The reusable and electrochemically active device 100 with the detachable electrode arrangement, holding at least the biological sample with at least the target bioanalyte, is connected to the point-of-care biosensor 300 through the device insertion port 305. The digital controller (307) through the signal conditioning and device detection unit (309), is adapted to detect and select at least an unused working electrode (101a, 101b, 101c, 101d, 101e, 101f) and an unused portion of the counter electrode (102) of the reusable and electrochemically active device (100) that is functionalized with an electrochemically active receptor corresponding to the at least target bioanalyte and facilitate loading of the at least biological sample with the at least target bioanalyte through corresponding unused fluid transportation channels (108a, 108b, 108c, 108d, 108e, 108f, 108g, 108h, 108i, 108j, 108k, 108l). The digital controller (307) through the signal conditioning and device detection unit (309) and voltage source (310), is also adapted to apply a redox potential and measure redox current from the at least unused working electrode through the current sensor (311) and display concentration levels of the at least target bioanalyte, the at least target bioanalyte, by correlating the measured redox current with a reference concentration of the at least target bioanalyte and to display the measured concentration level of the at least target bioanalyte on the display member (304), along with historical data.

Now, the preferred embodiments of the method for measuring the concentrations of target bioanalytes in biological samples, are now described by referring to FIG. 9. The desired biological samples such as blood are collected in very small volumes i.e., in the range of micro litres (μL), from human subjects, with a minimally invasive means, by following standard protocols. In the method of present invention, the preferred volume of the biological sample that can be used for the measurement of bioanalyte is preferably in the range of 1-10 micro litres (μL). The required volume of the biological sample is subject to the size of the capillaries dimensions of the device. The reduced collection of sample substantially reduces trauma in the subjects, since it is obtained through a minimally invasive sample extraction technique. The reduced volume of biological samples avoids the need for a user to phlebotomy collection products.

In the method of the present invention, the determination and accurate measurement of bioanalytes, in biological samples, is performed by implementing the principle of electrochemistry, by using the reusable electrochemical device 100 of the present invention.

In case the device holder 200 with the reusable electrochemical device 100 that is loaded with at least a biological sample with at least a target bioanalyte is selected for implementing the method of the present invention, the device holder 200 is advantageously connected to an external processing resource, for implementing the method of the present invention.

Whereas, in case, the reusable electrochemical device 100 that is loaded with at least a biological sample with at least a target bioanalyte, is selected for implementing the method of the present invention, the reusable electrochemical device 100 is advantageously connected to the point-of-care biosensor 300.

In the method of present invention, initially, the electrochemically active receptor substance that can interact with the desired target bioanalyte is prepared, advantageously as a solution of preferred substances, which can bind and/or react with target bioanalyte. The solution may be made with water or suitable solvents. The receptor solution thus prepared is introduced into the selected fluid transportation channels and from there transported to the electrode arrangement (working electrodes and counter electrode) of the reusable electrochemical device 100, prior to the application of biological samples with target bioanalytes.

Alternately, receptor solution can also be premixed with the biological samples with target bioanalytes and the mixed solution is introduced into the selected fluid transportation channels and from there transported to the electrode arrangement of the reusable electrochemical device 100 reusable electrochemical device 100.

In order to measure the presence of at least a target bioanalyte in biological samples, a reduced volume of the biological sample is brought in chemical contact with the functionalised electrode arrangement of the device 100.

Prior to the measurement of concentration of the target bioanalyte(s) in desired biological samples, such a blood, data pertaining to standard bioanalyte concentrations (g/dL) in various human blood samples are collected and stored in a database member of the point-of-care biosensor 300. Thus, the database member is populated with the values of standard bioanalyte concentrations (g/dL) along with the corresponding redox current values (μA). The preferred redox current values for the designated concentrations are obtained in an iterative manner, where repeated tests, result in identical redox current values, for the selected bioanalyte concentration. The measured redox current is matched with the stored redox current values and the matching bioanalyte concentration is secured and displayed by the point-of-care biosensor 300.

Alternately, the linear-fit equation (as shown in the examples) can also be used to compute the concentration of bioanalyte by using the redox current value. The point-of-care biosensor 300 after having extracted the value of concentration of the target bioanalyte(s) in the blood sample(s) displays the value. Therefore, the database member is populated with values of redox currents and concentration of desired bioanalytes in a known manner.

The reusable and electrochemically active device 100 is then connected to the point-of-care biosensor 300 or an external processing resource 205, as the case may be.

The point-of-care biosensor 300/processing resource 205 is then switched on to initiate the process of detecting the reusable and electrochemically active device 100.

The following steps of the method are now described in conjunction with the point-of-care biosensor 300. It is therefore understood that these process steps can also be suitably adapted for use with the external processing resource 205.

Once the reusable and electrochemically active device 100 is detected at least a detached electrode arrangement is identified. The identification of the detached electrode arrangement is performed by measuring a substantially zero current at the corresponding conducting tracks. The detached electrode arrangements are those, which were already used and detached from the reusable and electrochemically active device 100.

Thereafter, the next available at least unused and functionalised working electrode and counter electrode, is identified made available for introducing selected biological sample(s), with target bioanalyte(s).

The selected biological sample (blood) is taken in small quantity and introduced into an open end of at least one of the available the fluid transportation channels, that are in fluid communication with the selected working electrode and the counter electrode, by opening the sealing members, through any known means, such as micro-capillary pipet etc. Once the selected biological sample enters the selected fluid transportation channels, it is then transported to the at least unused and functionalised working electrode.

A redox potential is applied to the at least functionalised working electrode of the reusable and electrochemically active device 100 and the corresponding redox current is measured. Redox potential is a measure of the tendency of a chemical substance to acquire electrons and thereby be reduced. Each chemical substance has its own intrinsic redox potential. The more positive the potential, the greater is the substance affinity for electrons and the tendency to be reduced. The redox current that is passing through the counter and the at least working electrode is measured by using I to V converter.

The concentration levels of the target bioanalytes are measured by correlating the measured redox current with a reference concentration of the at least target bioanalyte and displayed. Alternately, the linear-fit equation can also be used to compute the concentration of the target bioanalytes by using the redox current value.

Once, the step of measurement and display of the concentration of the target bioanalyte is completed, the used working electrode(s), the fluid transportation channels and a partial portion of the counter electrode are detached from the reusable and electrochemically active device, either manually or by a tool.

Subsequent to the detachment of the used working electrode arrangement, the reusable and electrochemically active device is either stored for future use or used for additional measurement of the concentrations of desired bioanalytes.

Accordingly, the method of the present invention measuring the concentrations of target bioanalytes in biological samples, comprises the steps of: selecting the reusable and electrochemically active device and identifying at least an unused and functionalized working electrode, unused portion of a counter electrode and unused fluid transportation channel. The determination of the unused section or portion of electrode arrangement is performed by measuring a current, where the measured current is of a very low value. Conversely, the used electrode arrangement exhibits a very high current value. Once the available working electrode or electrodes are determined, at least a biological sample with at least a target bioanalyte is introduced into the at least unused fluid transportation channel of the unused working electrode. The biological sample is then transported to the at least unused and functionalized working electrode and the unused portion of the counter electrode, through a capillary action. Then, a redox potential is applied to the at least unused and functionalized working electrode and the unused portion of counter electrode and the corresponding redox current is measured. The concentration levels of the target bioanalyte(s) is measured by correlating the measured redox current with a reference concentration of the at least target bioanalyte. The measured concentrations of the target bioanalyte(s) are displayed. Once, the measurement of the concentrations of the target bioanalyte is competed the used portion of the electrode arrangement including at least the used working electrodes, at least a partial portion of the used counter electrode and the used fluid transportation channels are detached from the reusable and electrochemically active device.

The method of the present invention can be used for introduction of multiple biological samples with target bioanalytes at different unused fluid transportation channels, for transmission to respective unused and functionalised working electrodes.

In the method of the present invention, detection of the unused working electrodes is performed automatically by a voltage source and a current sensor.

The method of the present invention is now illustrated in the form of the following examples. These examples are provided for purpose of illustration and shall not be construed as limiting the scope of the invention.

The working electrodes of the reusable and electrochemically active device, is functionalized with an exemplary bioanalyte sensing, i.e., the electrochemically active and glucose-binding receptor, such that a single the reusable and electrochemically active device can measure more than one glucose bioanalyte in a biological sample (blood). The sensing chemistry for the electrochemically active and glucose-binding receptor is advantageously prepared as a solution of preferred chemical substances as hereinafter described. For instance, a combination of glucose oxidase as a capture molecule for glucose and potassium ferricyanide as a mediator molecule is selected as a preferred glucose sensing chemistry. It is understood here that other enzymatic or non-enzymatic glucose sensing chemistry can also be used for the electrochemically active and glucose-binding receptor. A microliter drop of receptor solution is introduced into the fluid transportation channel or channels to form a dry chemical layer of receptor, prior to the application of biological samples. Alternately, the receptor solution can also be premixed with the biological samples and the mixed solution is applied to the capillaries of the device. In order to test the presence of glucose bioanalyte in a blood sample, a reduced volume of the biological sample (whole blood) is brought in chemical contact with unused working electrodes of the device of the present invention. The method of the present invention can also be performed for measurement of other blood and/or urine biomarkers such as but not limited to proteins, peptides, enzymes and ions.

### Example 1: Determination of Glucose Concentrations in Two Different Whole Blood Samples Using Two Working Electrodes if the Reusable and Electrochemically Active Device

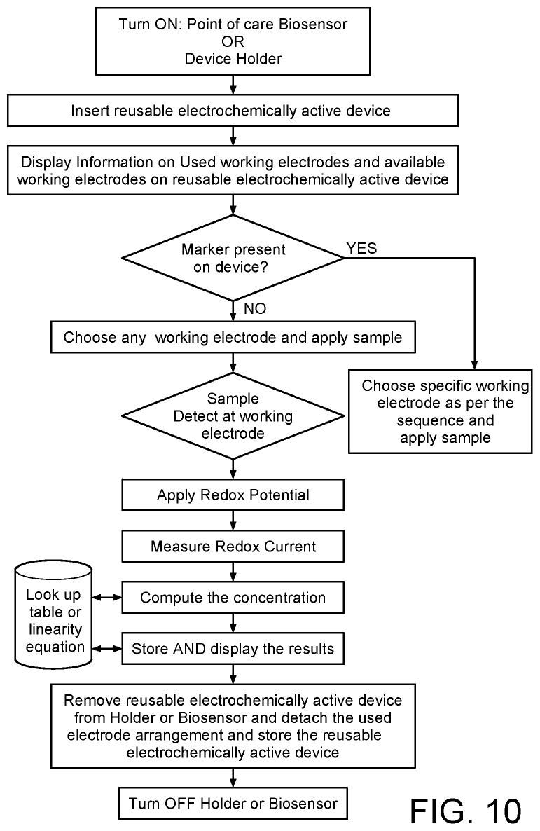

The reusable and electrochemically active device is connected to the point-of-care biosensor and the available unused working electrode(s) is determined. A master solution of glucose oxidase and potassium ferricyanide is prepared by dissolving the 50 mg potassium ferricyanide 10 ml of saline water. A master solution of sensing chemistry (electrochemically active and glucose binding receptor) is prepared by dissolving the 5 mg glucose oxidase in this 10 ml solution. The 1-10 μL drop of above solution is introduced into each of two unused fluid transportation channels of the reusable and electrochemically active device for transportation to the corresponding working electrodes (1 and 2) for their functionalization. Equilibration time given before running the process is 1 sec to 60 sec. A 1-5 μL volume of the human whole blood sample is taken and introduced into the fluid transportation channels for further transportation to the functionalised working electrodes (1 and 2). A redox voltage of 0.4V is applied to the selected functionalised working electrodes and the corresponding redox current is measured, from the functionalised working electrodes (1 and 2) upon reaction of the receptor with glucose bioanalyte of the whole blood sample. The redox current is observed to vary linearly with an increase in the concentrations of glucose in the whole blood sample at both the working electrodes, as shown in FIG. 10. The values of concentrations of glucose in blood plasma (mg/dL) along with corresponding redox current values (μA) are recorded and tabulated as shown in Table 1. The required data as shown in Table 1 are obtained from linear fit equation as given below:

y=0.0423x+1.4261

In the above equation, “y” represents the oxidation current value and “x” represents the concentration of analyte.

TABLE 1
Blood Plasma Glucose and corresponding redox currents

| | Oxidation | Oxidation |
|---|---|---|
| Blood plasma | current (μA) | current (μA) |
| glucose | Working | Working |
| (mg/dL) | Electrode-1 | Electrode-2 |
| 71 | 4.8 | 5.2 |
| 142 | 6.9 | 6.9 |
| 162 | 8.0 | 8.1 |
| 292 | 14.4 | 14.1 |

The above-stated process steps are repeated for another biological sample(s) by seeking the other available working electrodes. The used working electrode arrangement, including a partial portion of the counter electrode and the fluid transportation channels are detached from the reusable and electrochemically active device.

### Example 2: Determination of Concentration of Glucose Bioanalyte Using Four Working Electrodes of the Single Reusable the Reusable and Electrochemically Active Device

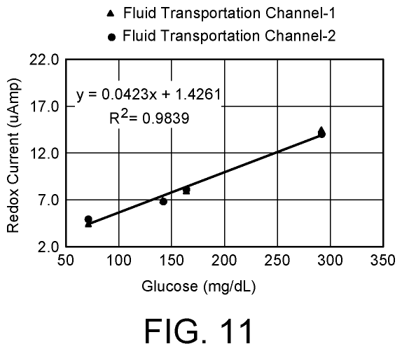

The reusable and electrochemically active device is connected to the point-of-care biosensor and the available unused working electrode(s) is determined. A master solution of glucose oxidase and potassium ferricyanide is prepared by dissolving the 50 mg potassium ferricyanide 10 ml of saline water. A master solution of sensing chemistry (electrochemically active and glucose binding receptor) is prepared by dissolving the 5 mg glucose oxidase in this 10 ml solution. The 0.1-10 μL drop of above solution is introduced into the fluid transportation channels for further transportation to each of the functionalised working electrodes (Four working electrodes). Equilibration time of 1 sec to 60 sec is given before running the experiment. A 1-5 μL volume of the human whole blood sample is taken and introduced into the corresponding fluid transportation channels for further transportation to four working electrodes. A redox voltage of 0.4V is applied to the selected four working electrodes and the corresponding redox current is measured on upon reaction with glucose bioanalyte of the blood samples. The oxidation current is observed to be varying linearly with an increase in the concentration of glucose in the whole blood sample, in the working electrodes as shown in FIGS. 11(a)-(d). The values of concentrations of the blood plasma glucose (mg/dL) along with corresponding reduction current values (μA) are recorded and tabulated as shown in Table 2 for suitable display. The required data as shown in Table 2 are obtained from different linear fit equations (different linearity equations), for the four working electrodes as given below:

y=0.1018x+10.118 Working Electrode-1

y=0.1052x+9.9647 Working Electrode-2

y=0.1111x+8.9442 Working Electrode-3

y=0.105x+8.5068 Working Electrode-4

In the above equations “y” represents the oxidation current value and “x” represents the concentration of the bioanalyte (glucose).

The above-stated process steps are repeated for another biological sample(s) by seeking the other available working electrodes. The used working electrode arrangement, including a partial portion of the counter electrode and the fluid transportation channels are detached from the reusable and electrochemically active device.

TABLE 2
Blood Plasma Glucose and corresponding
redox currents in 4 capillaries

| | Oxidation | Oxidation | Oxidation | Oxidation |
|---|---|---|---|---|
| Blood Plasma | current (μA) | current (μA) | current (μA) | current (μA) |
| Glucose | Working | Working | Working | Working |
| (mg/dL) | Electrode-1 | Electrode -2 | Electrode -3 | Electrode -4 |
| 99 | 20.19 | 22.48 | 20.53 | 20.65 |
| 106 | 19.91 | 23.81 | 23.31 | 18.88 |
| 178 | 28 | 29 | 26 | 25 |
| 337 | 43 | 46 | 44 | 44 |

### Example 3: Determination of Concentration of Glucose Concentration and Haemoglobin Using Two Working Electrodes on a on a Single Reusable and Electrochemically Active Device

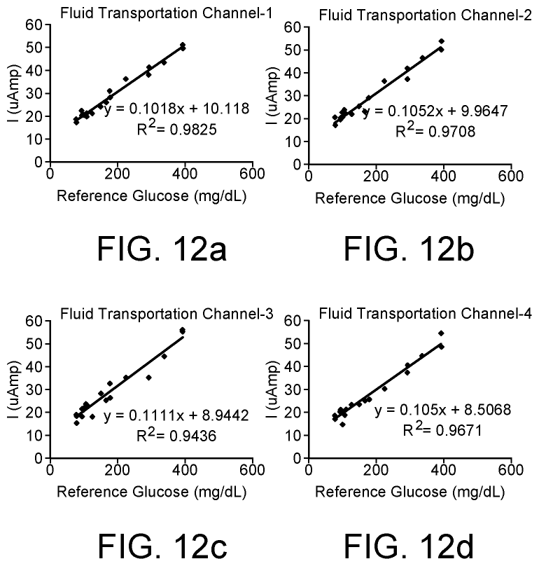

The reusable and electrochemically active device is connected to the point-of-care biosensor and the available unused working electrode(s) is determined. A master solution of glucose oxidase and potassium ferricyanide is prepared by dissolving the 50 mg potassium ferricyanide 10 ml of saline water. A master solution of sensing chemistry (electrochemically active and glucose binding receptor) is prepared by dissolving the 5 mg glucose oxidase in this 10 ml solution. A 0.1-10 μL drop of above solution is introduced into the fluid transportation channel 1 for transportation to the working electrode-1 of device and allowed to dry. A 0.1-10 μL drop of haemoglobin sensing IP solution is introduced into the fluid transportation channel-2 for transportation to the working electrode-2 of device allowed it to dry. A 1-5 μL volume of a human whole blood sample is taken and introduced into the fluid transportation channel-1 for transportation to the working electrode-1. A redox voltage of 0.4 is applied to the working electrode-1 and the corresponding redox current is measured on upon reaction with glucose bioanalyte of the blood sample and the concentration of the blood glucose analyte is measured. While the other whole blood sample is introduced into the fluid transportation channel 2 for transportation to the working electrode-2 and a redox voltage of 0.45 is applied to the working electrode-2 and the corresponding redox current is measured on upon reaction with haemoglobin bioanalyte of the blood sample and the concentration of the blood haemoglobin analyte is measured as shown in FIG. 12(a)-(b). The linear equation for both the bioanalytes (glucose and haemoglobin) is as given below:

For blood glucose:

y=0.0993x+10.374

For blood haemoglobin:

y=2.7772x+38.538

In the above equations “y” represents the oxidation current value and “x” represents the concentration of the corresponding bioanalytes.

The above-stated process steps are repeated for another biological sample(s) by seeking the other available working electrodes. The used working electrode arrangement, including a partial portion of the counter electrode and the fluid transportation channels are detached from the reusable and electrochemically active device.

### Advantages of the Present Invention

The marker or the sequence indicator, to indicate the commencement of sequence of introducing of biological samples into the fluid transportation channels for onward transportation to the respective electrodes, assists user in selecting a sequence to introduce biological samples through fluid transport channels for further transportation to the selected electrode arrangement.

In the device of present invention, the used electrode arrangement including working electrodes and a portion of the counter electrode and the fluid transportation channels are adapted to be detached from the reusable and electrochemically active device, after their use, to prevent a cross-contamination, while the remaining electrode arrangement is made available for the electro-chemical measurement of bioanalytes. The present invention also enables identification of the detached portion of the electrode arrangement to prevent a reuse of the used electrode arrangement.

The device and method of the present invention facilitates measurement of not only the concentrations of multiple target bioanalytes but also to measure, repeatedly, concentration of selected target bioanalyte, in biological samples.

1. A reusable and electrochemically active device (100), comprising:
(i) an electrode arrangement including working electrodes (101a, 101b) and a counter electrode (102) that are functionalized with an electrochemically active receptor corresponding to at least a target bioanalyte present in at least a biological sample and conducting tracks (103a, 103b, 103c), disposed on a substrate (104);
(ii) a spacer (105) disposed on the substrate (104), to overlay on the electrode arrangement;
(iii) openings (106a, 106b) are formed on the spacer (105) to expose at least portions of the functionalized working electrodes (101a, 101b) and the counter electrode (102);
(iv) a laminating member (107) disposed on the functionalized working electrodes (101a, 101b) and the counter electrode (102) such that the intervening spaces between the openings (106a, 106b) and the inner portion of the laminating member (107) form fluid transportation channels (108a, 108b) to receive the at least a biological sample with the at least a target bioanalyte and transport to the functionalized working electrodes (101a, 101b) and the counter electrode (102), by capillary action; and
(v) a voltage source (110) and a current sensor (111) are configured to be coupled to the electrode arrangement to apply a redox voltage and measure a redox current from the functionalized working electrodes (101a, 101b), upon interacting with the at least target bioanalyte, wherein the measured redox current is usable to obtain a concentration of the at least target bioanalyte, by correlating the measured redox current with a reference concentration of the at least target bioanalyte;
wherein the electrode arrangement including used working electrodes (101a, 101b), a partial portion of the counter electrode (102) and the fluid transportation channels (108a, 108b) are detachable, from the substrate (104), and
wherein insulating members (113a, 113b) are disposed on the conducting tracks (103a, 103b) such that the electrical connectivity and the electro-chemical integrity of the electrode arrangement, is retained even after the detachment of one of the used working electrodes (101a, 101b), along with a partial portion of the counter electrode (102) and one of the used fluid transportation channels (108a, 108b), from the reusable and electrochemically active device (100).

2. The device (100) as claimed in claim 1, wherein the electrode arrangement includes functionalized working electrodes (101a, 101b, 101c, 101d), counter electrode (102) that are in fluid communication with plurality of fluid transportation channels (108a, 108b, 108c, 108d), and insulating members (113a, 113b, 113c, 113d) are disposed on the conducting tracks (103b, 103d, 103e), such that the electro-chemical integrity of the electrode arrangement is retained even after detachment at least one of the used working electrodes (101a, 101b, 101c, 101d), partial portions of the counter electrode (102) and at least one of the used fluid transportation channels (108a, 108b, 108c, 108d), from the reusable and electrochemically active device (100).

3. The device (100) as claimed in claim 1, wherein a marker or a sequence indicator (114) is disposed at a pre-determined working electrode, to indicate the commencement of sequence of introducing of biological samples into the fluid transportation channels (108a, 108b, 108c, 108d) for onward transportation to the respective electrodes (101a, 101b, 101c, 101d).

4. The device (100) as claimed in claim 2, wherein a marker or a sequence indicator (114) is disposed at a pre-determined working electrode, to indicate the commencement of sequence of introducing of biological samples into the fluid transportation channels (108a, 108b, 108c, 108d) for onward transportation to the respective electrodes (101a, 101b, 101c, 101d).

5. The device (100) as claimed in claim 1, wherein the fluid transportation channels (108a, 108b, 108c, 108d) that are in fluid communication with the functionalized working electrodes (101a, 101b, 101c, 101d) are with horizontal, angular and vertical orientations.

6. The device (100) as claimed in claim 2, wherein the fluid transportation channels (108a, 108b, 108c, 108d) that are in fluid communication with the functionalized working electrodes (101a, 101b, 101c, 101d) are with horizontal, angular and vertical orientations.

7. The device (100) as claimed in claim 1, wherein including a set of fluid transportation channels (108e, 108f, 108g, 108h) in fluid communication with the functionalized working electrode (101e), a set of fluid transportation channels (108i, 108j, 108k, 108l) in fluid communication with the functionalized working electrode (101f), a counter electrode (102) disposed in between the functionalized working electrodes (101e, 101f) and an insulating member (103e) disposed on the conducting track (103b).

8. The device (100) as claimed in claim 1, wherein sealing members (109a, 109b, 109c, 109d) are connected to the fluid transportation channels (108a, 108b, 108c, 108d).

9. The device (100) as claimed in claim 2, wherein sealing members (109a, 109b, 109c, 109d) are connected to the fluid transportation channels (108a, 108b, 108c, 108d).

10. The device (100) as claimed in claim 8, wherein sealing members (109e, 109f, 109g, 109h, 109i, 109j, 109k, 109l) are connected to the fluid transportation channels (108e, 108f, 108g, 108h, 108i, 108j, 108k, 108l).

11. The device (100) as claimed in claim 1, wherein the at least target bioanalyte is selected from glucose, proteins, peptides, enzymes, antigens and antibodies.

12. The device (100) as claimed in claim 1, wherein the electrochemically active receptor is interactive with the at least target bioanalyte.

13. The device (100) as claimed in claim 12, wherein the electrochemically active receptor is interactive with the at least target bioanalyte.

14. The device as claimed in claim 1, wherein the voltage source (110) and the current sensor (111) are disposed and adapted to automatically detect the used and unused working electrodes (101a, 101b, 101c, 101d) and the corresponding sections of counter electrode (102) of the electrochemically active device (100).

15. The device as claimed in claim 2, wherein the voltage source (110) and the current sensor (111) are disposed and adapted to automatically detect the used and unused working electrodes (101a, 101b, 101c, 101d) and the corresponding sections of counter electrode (102) of the electrochemically active device (100).

16. A device holder (200) for holding the reusable and electrochemically active device (100), the device holder (200) comprising:
(i) a holder housing (201) including a device detection and signal conditioning module;
(ii) a USB connector (203) is adapted to be connected to an external electronic processing resource (205); and
(iii) the reusable and electrochemically active device (100) is adapted to be connected to a device insertion port (204) and to an external electronic processing resource (205) and the reusable and electrochemically active device (100) is adapted to receive at least a biological sample with at least a target bioanalyte, through at least an unused fluid transportation channels (108a, 108b, 108c, 108d, 108e, 108f, 108g, 108h, 108i, 108j, 108k, 108l), and transported to at least an unused working electrode (101a, 101b, 101c, 101d, 101e, 101f) and unused portion of the counter electrode (102), after establishing a connectivity with the external electronic processing resource (205).

17. The holder (200) as claimed in claim 16, wherein the external electronic processing resource (205) is at least one of a hand-held computing device or a communicating device with a processor.

18. The holder (200) as claimed in claim 16, wherein the at least biological sample is blood or urine.

19. A point-of-care biosensor (300) for measuring concentrations of target bioanalytes in biological samples, comprising:
(i) a micro USB (302), a micro SD card (303), a display member (304), a device insertion port (305), a database member (306), a voltage source (310) and a current sensor (311), a signal conditioning and device detection unit (309), humidity and temperature sensors (310, 311) are adapted to be connected to a digital controller (307) and disposed in a sensor housing (301) and connected to a power supply unit (308); and
(ii) the reusable and electrochemically active device (100) with the detachable electrode arrangement, holding at least a biological sample with at least a target bioanalyte, is connected to the point-of-care biosensor (300) through the device insertion port (305);
wherein, the digital controller (307) through the signal conditioning and device detection unit (309), is adapted to detect and select at least an unused working electrode (101a, 101b, 101c, 101d, 101e, 101f) and an unused portion of the counter electrode (102) of the reusable and electrochemically active device (100) that is functionalized with an electrochemically active receptor corresponding to the at least target bioanalyte and facilitate loading of the at least biological sample with the at least target bioanalyte through corresponding unused fluid transportation channels (108a, 108b, 108c, 108d, 108e, 108f, 108g, 108h, 108i, 108j, 108k, 108l), and
wherein the digital controller (307) through the signal conditioning and device detection unit (309) and voltage source (310), is adapted to apply a redox potential and measure redox current from the at least unused working electrode through the current sensor (311) and display concentration levels of the at least target bioanalyte, the at least target bioanalyte, by correlating the measured redox current with a reference concentration of the at least target bioanalyte and to display the measured concentration level of the at least target bioanalyte on the display member (304), along with historical data.

20. The point-of-care biosensor (300), as claimed in claim 19, wherein the database member (306) includes stored standard values of redox current and corresponding concentrations of the at least target bioanalyte along with the historical data.

21. A method for measuring the concentrations of target bioanalytes in biological samples, the method comprising the steps of:
(a) selecting a reusable and electrochemically active device;
(b) identifying at least an unused and functionalized working electrode, unused portion of a counter electrode and unused fluid transportation channel;
(c) introducing at least a biological sample with at least a target bioanalyte into the at least unused fluid transportation channel and transporting through a capillary action to the at least unused and functionalized working electrode and the unused portion of the counter electrode;
(d) applying a redox potential to the at least unused and functionalized working electrode and the unused portion of counter electrode and measuring the corresponding redox current;
(e) measuring and displaying concentration levels of the at least target bioanalyte, by correlating the measured redox current with a reference concentration of the at least target bioanalyte; and
(f) detaching only the used portion of the electrode arrangement including at least the used working electrodes, at least a partial portion of the used counter electrode and the used fluid transportation channels, from the reusable and electrochemically active device.

22. The method as claimed in claim 21, wherein multiple biological samples with target bioanalytes are collected at different unused fluid transportation channels, for transmission to respective unused and functionalized working electrodes.

23. The method as claimed in claim 21, wherein including a step of selecting at least an electrochemically active receptor based on its interactive capability with the desired target bioanalyte.

24. The method as claimed in claim 21, wherein the step of detaching the used working electrode is preferably performed by a tool or by hand.

25. The method as claimed in claim 21, wherein the detection of the unused working electrodes is performed automatically by a voltage source and a current sensor.
